(19) United States
(12) Patent Application Publication
(10) Pub. No.: US 2012/0277689 A1
(43) Pub. Date: Nov. 1, 2012

(54) QUATERNARY NICKEL-TITANIUM ALLOY

(75) Inventors: James M. Carlson, Bloomington, IN (US); Mark A. Magnuson, Bloomington, IN (US)

(73) Assignee: Cook Medical Technologies LLC, Bloomington, IN (US)

(21) Appl. No.: 13/457,824

(22) Filed: Apr. 27, 2012

Related U.S. Application Data
(60) Provisional application No. 61/480,855, filed on Apr. 29, 2011.

(51) Int. Cl. C22C 19/05; A61M 25/00; C22C 30/00

(52) U.S. Cl. 604/264; 420/451; 420/588

(57) ABSTRACT

A quaternary nickel-titanium alloy includes: Ni at a concentration of between about 48 at. % and about 52 at. %; Cr at a concentration of from about 0.3 at. % to about 1 at. %; Co at a concentration of from about 0.5 at. % to about 2 at. %; and Ti at a concentration wherein a ratio of Ni:Ti is about 1.03. According to one exemplary embodiment of the alloy, the concentration of Cr may be about 0.5 at. % and the concentration of Co may be about 0.75 at. %. According to another exemplary embodiment of the alloy, the concentration of Cr may be about 0.25 at. % and the concentration of Co may be about 0.5 at. %.

### RELATED APPLICATION

The present patent document claims the benefit of the filing date under 35 U.S.C. §119(e) of U.S. Provisional Patent Application Ser. No. 61/480,855, filed on Apr. 29, 2011, which is hereby incorporated by reference in its entirety.

### TECHNICAL FIELD

The present disclosure is directed generally to nickel-titanium alloys and more particularly to a quaternary nickel-titanium alloy including cobalt and chromium as additional alloying elements to achieve improved mechanical and superelastic properties.

### BACKGROUND

Nickel-titanium alloys are commonly used for the manufacture of endoluminal biomedical devices, such as self-expandable stents, stent grafts, embolic protection filters, and stone extraction baskets. These devices may exploit the superelastic or shape memory behavior of equiatomic or near-equiatomic nickel-titanium alloys. Such alloys, which are commonly referred to as Nitinol or Nitinol alloys, undergo a phase transformation between a lower temperature phase (martensite) and a higher temperature phase (austenite) that allows a previous shape or configuration to be “remembered” and recovered.

For example, strain introduced into a Nitinol stent in the martensitic phase to achieve a compressed configuration may be substantially recovered upon completion of a reverse phase transformation to austenite, allowing the alloy to elastically spring back to an expanded configuration. The strain recovery may be driven by the removal of an applied stress (superelastic effect) and/or by a change in temperature (shape memory effect). Typically, strains of up to 8-10% may be recovered during the phase transformation.

Some nickel-titanium shape memory alloys may exhibit a two-stage transformation which includes a transformation to a rhombohedral phase (R-phase) in addition to the monoclinic (B12) martensitic phase and the cubic (B2) austenitic phase. The transformation to R-phase in two-stage shape memory materials occurs prior to the martensitic transformation upon cooling and prior to the austenitic transformation upon heating.

As generally understood by those skilled in the art, martensite start temperature (Mₛ) refers to the temperature at which the phase transformation to martensite begins upon cooling, and martensite finish temperature (M_{f}) refers to the temperature at which the phase transformation to martensite concludes. Austenite start temperature (Aₛ) refers to the temperature at which the phase transformation to austenite begins upon heating, and austenite finish temperature (A_{f}) refers to the temperature at which the phase transformation to austenite concludes. R-phase start temperature (Rₛ) refers to the temperature at which a phase transformation to R-phase begins upon cooling for a two-stage shape memory material, and R-phase finish temperature (R_{f}) refers to the temperature at which the phase transformation to R-phase concludes upon cooling. Finally, R′-phase start temperature (R′ₛ) is the temperature at which a phase transformation to R-phase begins upon heating for a two-stage shape memory material, and R′-phase finish temperature (R′_{f}) is the temperature at which the phase transformation to R-phase concludes upon heating.

For some medical device applications (e.g., stents employed in the superficial femoral artery (SFA)), an enhancement of the properties of conventional binary Nitinol alloys is desired. For example, due to its location in the vicinity of the hip joint, the SFA may experience repetitive axial strains that can cause the artery to elongate or contract up to 10-12%. Stents placed in the SFA may thus be prone to fatigue failure. In addition, a stent deployed in the SFA or other superficial arteries may be subjected to crushing loads due to the proximity of the artery to the surface of the skin. A major challenge of treating the SFA is providing a stent having sufficient elasticity, crush resistance, and fatigue properties to withstand the strains of the arterial environment.

### BRIEF SUMMARY

A quaternary nickel-titanium alloy including cobalt and chromium as alloying elements and exhibiting favorable superelastic and mechanical properties is set forth herein. Also described is a medical device comprising the quaternary nickel-titanium alloy.

The quaternary nickel-titanium alloy includes Ni at a concentration of from about 48 at. % to about 52 at. %; Cr at a concentration of from about 0.3 at. % to about 1 at. %; Co at a concentration of from about 0.5 at. % to about 2 at. %; and Ti at a concentration wherein a ratio of Ni:Ti is about 1.03.

The medical device includes at least one component comprising the quaternary nickel-titanium alloy, which may include Ni at a concentration of from about 48 at. % to about 52 at. %; Cr at a concentration of from about 0.3 at. % to about 1 at. %; Co at a concentration of from about 0.5 at. % to about 2 at. %; and Ti at a concentration wherein a ratio of Ni:Ti is about 1.03.

According to one embodiment of the quaternary nickel-titanium alloy, the concentration of Cr may be about 0.5 at. %, and the concentration of Co may be about 0.75 at. %.

According to another embodiment of the quaternary nickel-titanium alloy, the concentration of Cr may be about 0.25 at. %, and the concentration of Co may be about 0.5 at. %.

### BRIEF DESCRIPTION OF THE DRAWINGS

FIG. 1 is a plot of upper plateau strength (UPS) as a function of alloy composition for the tested binary Nitinol, Ni—Ti—Cr, Ni—Ti—Co, Ni—Ti—Fe and Ni—Ti—Co—Cr alloy samples, where the x-axis shows the atomic percentage of the alloying element(s) “Xx” (where Xx═Cr, Co, Fe, or Co—Cr) or, in the case of binary nickel-titanium, the atomic percentage of Ni above 49 at. %;

FIG. 2 is a plot of lower plateau strength (LPS) as a function of alloy composition for the tested binary Nitinol, Ni—Ti—Cr, Ni—Ti—Co, Ni—Ti—Fe and Ni—Ti—Co—Cr alloy samples, where the x-axis shows the atomic percentage of the alloying element(s) “Xx” (where Xx═Cr, Co, Fe, or Co—Cr) or, in the case of binary nickel-titanium, the atomic percentage of Ni above 49 at. %;

FIG. 3 is a plot of hysteresis as a function of alloy composition for the tested binary Nitinol, Ni—Ti—Cr, Ni—Ti—Co, Ni—Ti—Fe and Ni—Ti—Co—Cr alloy samples, where the x-axis shows the atomic percentage of the alloying element(s) “Xx” (where Xx═Cr, Co, Fe, or Co—Cr) or, in the case of binary nickel-titanium, the atomic percentage of Ni above 49 at. %;

FIG. 4 is a plot of permanent set as a function of alloy composition for the tested binary Nitinol, Ni—Ti—Cr, Ni—Ti—Co, Ni—Ti—Fe and Ni—Ti—Co—Cr alloy samples, where the x-axis shows the atomic percentage of the alloying element(s) “Xx” (where Xx═Cr, Co, Fe, or Co—Cr) or, in the case of binary nickel-titanium, the atomic percentage of Ni above 49 at. %;

FIG. 5 is a plot of uniform elongation as a function of alloy composition for the tested binary Nitinol, Ni—Ti—Cr, Ni—Ti—Co, Ni—Ti—Fe and Ni—Ti—Co—Cr alloy samples, where the x-axis shows the atomic percentage of the alloying element(s) “Xx” (where Xx═Cr, Co, Fe, or Co—Cr) or, in the case of binary nickel-titanium, the atomic percentage of Ni above 49 at. %; and

FIG. 6 is a plot of ultimate tensile strength (UTS) as a function of alloy composition for the tested binary Nitinol, Ni—Ti—Cr, Ni—Ti—Co, Ni—Ti—Fe and Ni—Ti—Co—Cr alloy samples, where the x-axis shows the atomic percentage of the alloying element(s) “Xx” (where Xx═Cr, Co, Fe, or Co—Cr) or, in the case of binary nickel-titanium, the atomic percentage of Ni above 49 at. %.

### DETAILED DESCRIPTION

Described here is a quaternary nickel-titanium alloy having improved superelastic and mechanical properties. The optimized alloy composition was derived from a series of experiments carried out on binary (Ni—Ti) and ternary (Ni—Ti—X) Nitinol alloy specimens. The experiments enabled the inventors to identify the most promising alloying elements and concentrations for a quaternary alloy composition, as well as a preferred nickel-to-titanium ratio.

Studied were three binary Nitinol alloys having differing Ni:Ti ratios, and nine ternary Nitinol alloys including either Cr, Co or Fe as a ternary alloying element at varying concentrations. In addition, data were obtained for two quaternary Nitinol alloys including both Cr and Co as alloying elements. As would be understood by one of ordinary skill in the art, binary Nitinol includes about 45-55 at. % Ni and about 45-55 at. % Ti and no additional alloying elements, with the exception of any incidental impurities. A ternary (or quaternary) Ni—Ti alloy includes one (or two) additional alloying element(s) in addition to nickel, titanium, and any incidental impurities. The alloy compositions, which are compiled in Table 1 below, were melted and drawn into wire by Fort Wayne Metals (Fort Wayne, Ind.). Materials certifications were received for each alloy sample to confirm the composition.

The ternary and quaternary drawn wire specimens included from about 38-42% cold work, where cold work refers to plastic deformation imparted to a component without applying heat, and percent (%) cold work provides a measurement of the amount of the plastic deformation, where the amount is calculated as a percent reduction in a given dimension. For example, in wire drawing, the percent cold work corresponds to the percent reduction in the cross-sectional area of the wire resulting from a drawing pass.

TABLE 1
Composition of Nitinol Specimens in Terms of Atomic Percent (%)

| | | |
|---|---|---|
| Alloy | Atomic % | % Coldwork |

| | | | |
|---|---|---|---|
| NiTi (low Ni) | 49.75 | (Ni) | 45.9 |
| NiTi (med. Ni) | 50.73 | (Ni) | 46.6 |
| NiTi (high Ni) | 51.25 | (Ni) | 46.2 |
| NiTiCr | 0.25 | (Cr) | 45 |
| NiTiCr | 0.5 | (Cr) | 38/42 |
| NiTiCr | 1.0 | (Cr) | 38/42 |
| NiTiCo | 0.5 | (Co) | 38/42 |
| NiTiCo | 1.0 | (Co) | 38/42 |
| NiTiCo | 2.0 | (Co) | 38/42 |
| NiTiFe | 2.0 | (Fe) | 38/42 |
| NiTiFe | 3.5 | (Fe) | 38/42 |
| NiTiFe | 5.0 | (Fe) | 38/42 |

| | | |
|---|---|---|
| NiTiCoCr | 0.5 (Co), 0.25 (Cr) | 38/42 |
| NiTiCoCr | 0.75 (Co), 0.5 (Cr) | 38/42 |

To carry out the analysis, tensile tests and differential scanning calorimetry (DSC) experiments were performed on the binary, ternary and quaternary nickel-titanium alloy wire specimens described in Table 1. The resulting tensile test and DSC data were analyzed to identify optimal amounts of Cr, Co and Ni for a “designer” quaternary Ni—Ti alloy that may exhibit an ideal combination of superelastic and mechanical properties as well as a suppressed martensite start temperature. The two different quaternary alloys tested (one including 0.75 at. % Co, and 0.5 at. % Cr; the other including 0.5 at. % Co and 0.25 at. % Cr) were selected based on the results obtained for the binary and ternary nickel-titanium alloys.

Using the collected data, the inventors identified the binary and ternary alloy compositions at which the mechanical and superelastic properties are optimized. The properties deemed to be of greatest importance in determining a preferred quaternary alloy composition include, along with suitable transformation temperatures:

(1) upper plateau strength;

(2) hysteresis;

(3) permanent set;

(4) elongation; and

(5) ultimate tensile strength.

Differential Scanning calorimetry (DSC) Experiments

DSC experiments were carried out on the wire samples to identify phase transformation temperatures. The DSC test method involves heating and cooling a test specimen at a controlled rate in a controlled environment through the temperature intervals of the phase transformations. The difference in heat flow between the test material and a reference due to energy changes is continuously monitored and recorded. Absorption of energy due to a phase transformation in the specimen results in an endothermic valley on heating. Release of energy due to a phase transformation in the specimen results in an exothermic peak upon cooling. Phase transformation temperatures (e.g., Mₛ, M_{f}, Rₛ, R_{f}, etc.) can be obtained from the DSC data by determining the start and finish of each transformation. Conventional DSC testing as prescribed in ASTM Standard F2004-05 or “double loop” DSC testing as set forth in U.S. patent application Ser. No. 12/274,556, which is hereby incorporated by reference in its entirety, was employed to for the experiments.

Of particular interest was identifying wire specimens having a suppressed martensite start (Mₛ) temperature. The inventors believe that an alloy having a reduced Mₛ temperature may have better fatigue life and exhibit a higher radial force than an alloy with a higher Mₛ temperature. The inventors also believe that appropriate alloying additions may lead to the formation of second phase precipitates in nickel-titanium alloys that reduce the probability of martensite formation during cooling or the application of a stress.

TABLE 2
Results of DSC Experiments

| Alloy/condition | Atomic % | Ms (° C.) | Mf (° C.) | R′s (° C.) | R′f (° C.) | Rs (° C.) | Rf (° C.) | As (° C.) | Af (° C.) |
|---|---|---|---|---|---|---|---|---|---|
| NiTi/strain aged | 49.75% | 44.4 | 30.2 | 78.5 | 86.9 | 63.6 | 57.5 | 94.5 | 106.2 |
| NiTi/strain aged | 50.73% | 18.6 | −7.9 | N/A | N/A | N/A | N/A | 2.5 | 21 |
| NiTi/strain aged | 51.25% | 23.2 | 8.1 | −16.2 | /0.8 | ? | ? | 12.2 | 24.1 |
| NiTiCr/45% CW & | 0.25% | 22 | 7 | | | | | 15 | 24.1 |
| 4500 C. for 20 min. | | (as-drawn - | (as- | | | | | (as- | (as-drawn - |
| | | 10.8) | drawn - | | | | | drawn - | 12.3) |
| | | | 14.9) | | | | | −10.4) | |
| NiTiCr/as cast | 0.5% | −30 cert | −63 cert | | | | | −25 cert | −11 cert |
| NiTiCr/strain aged | 0.5% | −101.2 | −127.9 | −32.2 | −15.5 | 13.4 | −10.4 | .6 | 15.5 |
| NiTiCr/as cast | 1.0% | −83 cert | −127 cert | | | | | −88 cert | −50 cert |
| NiTiCr/strain aged | 1.0% | −87.1 | −133.4 | −22.6 | −4.9 | 17.5 | −4.6 | 4.6 | 19.4 |
| NiTiCo/as cast | 0.5% | −19 cert | −53 cert | | | | | −26 cert | 0 cert |
| NiTiCo/strain | 0.5% | 3.7 | −26.8 | N/A | N/A | N/A | N/A | −18.6 | 6.6 |
| aged | | | | | | | | | |
| NiTiCo/as cast | 1.0% | −34 cert | −67 cert | | | | | −34 cert | −10 cert |
| NiTiCo/strain | 1.0% | −95.4 | −130.3 | −29.8 | −11.6 | 15.8 | −9.1 | 2.2 | 18.2 |
| aged | | | | | | | | | |
| NiTiCo/strain | 2.0% | 3.5 | −25.4 | N/A | N/A | N/A | N/A | −13.9 | 6.1 |
| aged | | | | | | | | | |
| NiTiFe/strain | 2.0% | −14.3 | −54 | N/A | N/A | N/A | N/A | −45.9 | −4.2 |
| aged | | | | | | | | | |
| NiTiCoCr/strain | 1.25% | −9.7 | −45.4 | N/A | N/A | N/A | N/A | −37.4 | −3.2 |
| aged | | | | | | | | | |
| NiTiCoCr/strain | 0.75% | −5.6 | −56.3 | N/A | N/A | N/A | N/A | −70 | 4 |
| aged | | | | | | | | | |

DSC of the binary Nitinol samples revealed a “sweet spot” for the desired nickel to titanium ratio. If the nickel content of the binary alloy is too low, the Mₛ temperature may not be suppressed as desired. If the nickel content is too high, the suppression may be so excessive that no transformation from R-phase to martensite is observed during DSC experiments. Based on the results of the experiments, the inventors believe that a nickel to titanium (Ni:Ti) atomic ratio of 1.025 is preferred. The DSC data also show that both Cr and Co additions depress the martensite start and martensite finish temperatures.

Also of interest is the austenite finish temperature (A_{f}) of the alloy. It is important for A_{f} to be sufficiently below body temperature (37° C.) to ensure that the alloy behaves superelastically if used in the body as part of a medical device. As evidenced in the above table, all of the nickel-titanium alloys including Cr or Co additions exhibit A_{f} values below 20° C. This is a sufficiently low starting point for the Nitinol such that when finished devices are manufactured and aged the A_{f} remains well under body temperature. Additionally, the farther A_{f} is from body temperature, the higher the magnitude of the radial force that will be achieved when body temperature is reached.

### Tensile Tests

Tensile tests of the wire specimens were carried out by Fort Wayne Metals (Fort Wayne, Ind.) per ASTM Standard F2516. A total of 53 tensile tests were performed on wire specimens of the 11 alloy compositions indicated in Table 3. The tests were used to reveal superelastic behavior as well as to provide standard mechanical properties data.

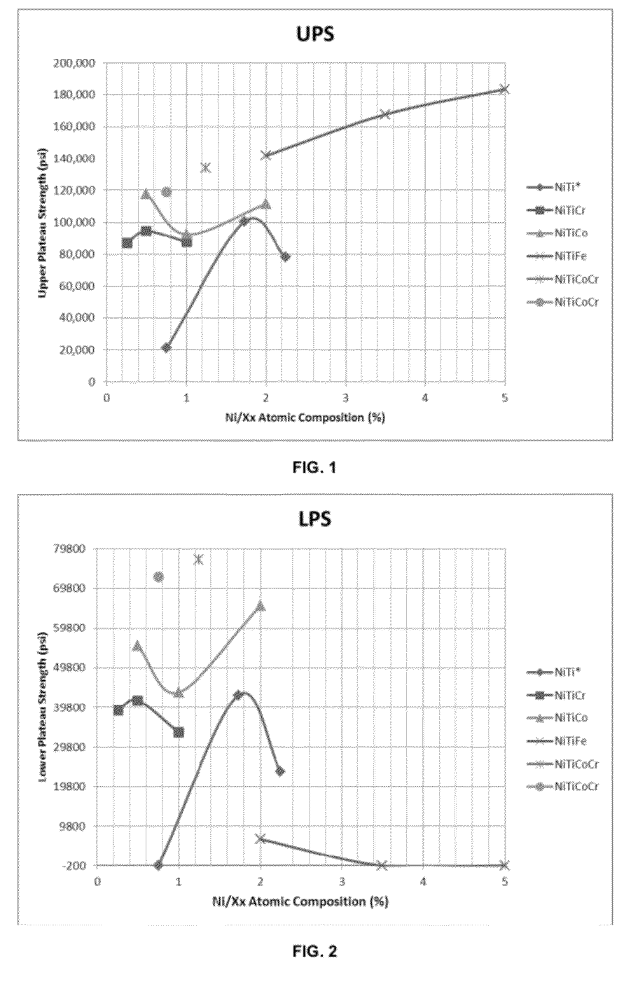

The average data obtained in Table 3 for each sample are plotted in FIGS. 1 through 6. The figures show, for the tested binary Nitinol, Ni—Ti—Cr, Ni—Ti—Co, Ni—Ti—Fe and Ni—Ti—Co—Cr alloy samples, how the measured properties vary as a function of alloy composition. In the case of the binary Ni—Ti alloy samples, the x-axis indicates the atomic percentage of nickel above 49 at. % that is present in the sample. For the ternary Ni—Ti—X (X═Cr, Co, or Fe) alloy samples, the x-axis indicates the atomic percentage of the ternary element present in the sample. For the quaternary Ni—Ti—Co—Cr alloy samples, the x-axis indicates the atomic percentage of the ternary and quaternary elements present in the sample.

FIG. 1 is a plot of upper plateau strength (UPS) as a function of alloy composition for the tested binary Ni—Ti, Ni—Ti—Cr, Ni—Ti—Co, Ni—Ti—Fe and Ni—Ti—Co—Cr alloy samples, where the UPS is averaged over five measurements for each sample. Upper plateau strength may be defined as the stress at 3% strain during loading of a superelastic nickel-titanium alloy specimen undergoing a tensile test in accordance with ASTM Standard F2516. This superelastic material property may be used as an indicator of the radial force that can be obtained from an expandable stent in use in a body vessel.

FIG. 1 shows that binary Ni—Ti exhibits a maximum UPS of about 101 ksi at a nickel concentration of about 50.7 at. % Referring to the data for the Ni—Ti—Cr alloy samples, it can be seen that the maximum UPS of about 95 ksi is achieved at a Cr concentration of about 0.5 at. %. For the Ni—Ti—Co alloy samples, the maximum UPS of about 118 ksi is achieved at a Co concentration of about 0.5 at. %. The high UPS achieved for the Ni—Ti—Fe alloy samples is somewhat diminished in value due to the excessively depressed transformation temperatures measured for these samples. High values of UPS (about 120 ksi and 134 ksi) are achieved for the two Ni—Ti—Co—Cr specimens.

FIG. 2 is a plot of lower plateau strength (LPS) as a function of alloy composition for the tested binary Ni—Ti, Ni—Ti—Cr, Ni—Ti—Co, Ni—Ti—Fe and Ni—Ti—Co—Cr alloy samples, where the LPS is averaged over several measurements for most of the samples. Lower plateau strength may be defined as the stress at 2.5% strain during unloading of a superelastic nickel-titanium alloy specimen, after loading to 6% strain during a tensile test in accordance with ASTM Standard F 2516.

The binary Ni—Ti alloy samples show a maximum LPS of about 43 ksi at about 50.7 at. % Ni. Referring to the data for the Ni—Ti—Cr and Ni—Ti—Co alloy samples, it can be seen that the maximum LPS is achieved at a concentration of about 0.25 at. % Cr (about 46 ksi) for the Ni—Ti—Cr alloy samples and 2.0 at. % Co (about 65 ksi) for the Ni—Ti—Co alloy samples, respectively. Unexpectedly high values of LPS (about 73 ksi and about 77 ksi) are obtained for the two Ni—Ti—Co—Cr alloy samples.

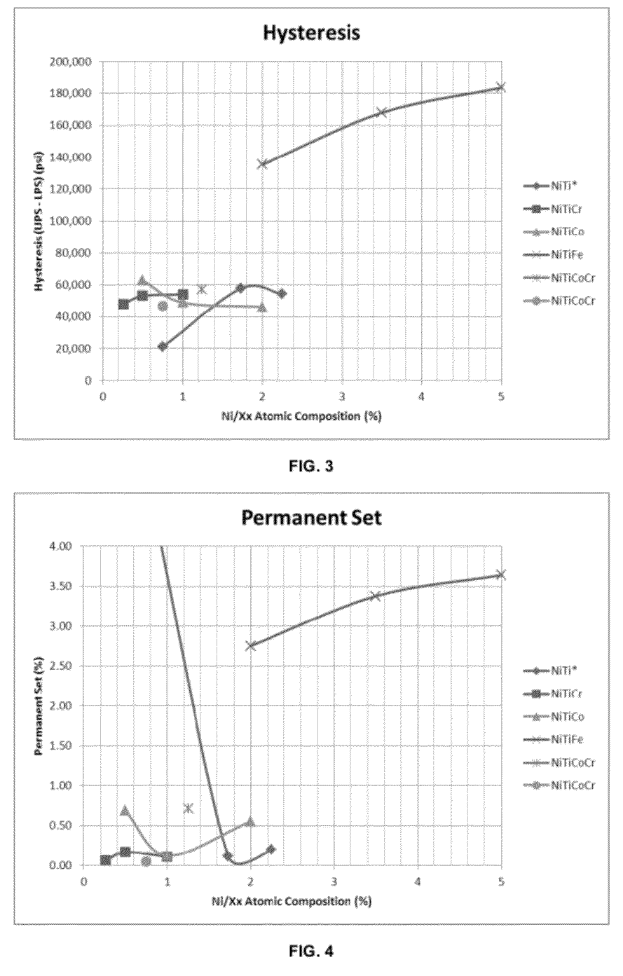

FIG. 3 is a plot of hysteresis as a function of alloy composition for the tested binary Ni—Ti, Ni—Ti—Cr, Ni—Ti—Co, Ni—Ti—Fe and Ni—Ti—Co—Cr alloy samples. Hysteresis may be defined as the difference between the upper plateau strength and the lower plateau strength.

The binary Ni—Ti alloys samples show a maximum value of hysteresis (58 ksi) at a nickel concentration of about 50.7 at. %. In the case of the Ni—Ti—Co alloy samples, the maximum hysteresis of about 63 ksi occurs at a Co concentration of about 0.5 at. % In the case of the Ni—Ti—Cr alloy samples, the maximum hysteresis of about 53-54 ksi occurs over a Cr concentration range of about 0.5 at. % to about 1 at. %, which represents a small decrease compared to the binary samples. The behavior of the quaternary Ni—Ti—Co—Cr samples is similar.

FIG. 4 is a plot of permanent set as a function of alloy composition for the tested binary Ni—Ti, Ni—Ti—Cr, Ni—Ti—Co, Ni—Ti—Fe, and Ni—Ti—Co—Cr alloy samples. Permanent set may be defined as the amount of strain (%) that remains in the specimen after removal of the applied stress. For a superelastic specimen, it is advantageous to minimize permanent set and maximize the amount of recoverable strain (i.e., nonpermanent strain that is recovered after an applied stress is removed).

Referring to the Ni—Ti—Cr data in FIG. 4, the permanent set is below 0.2% over a Cr concentration range of about 0.25 at. % to 1 at. %. In the case of the Ni—Ti—Co alloy samples, the lowest value of permanent set (0.12%) occurs at a Co concentration of about 1 at. %. For binary nickel-titanium, the permanent set is excessive without a sufficient amount of excess nickel (e.g., at least about 50.7 at. %). The quaternary sample including 0.5 at. % Co and 0.25 at. % Cr exhibits an almost negligible permanent set (about 0.05%) while the quaternary sample including 0.75 at. % Co and 0.5 at. % Cr has a higher permanent set of about 0.7%.

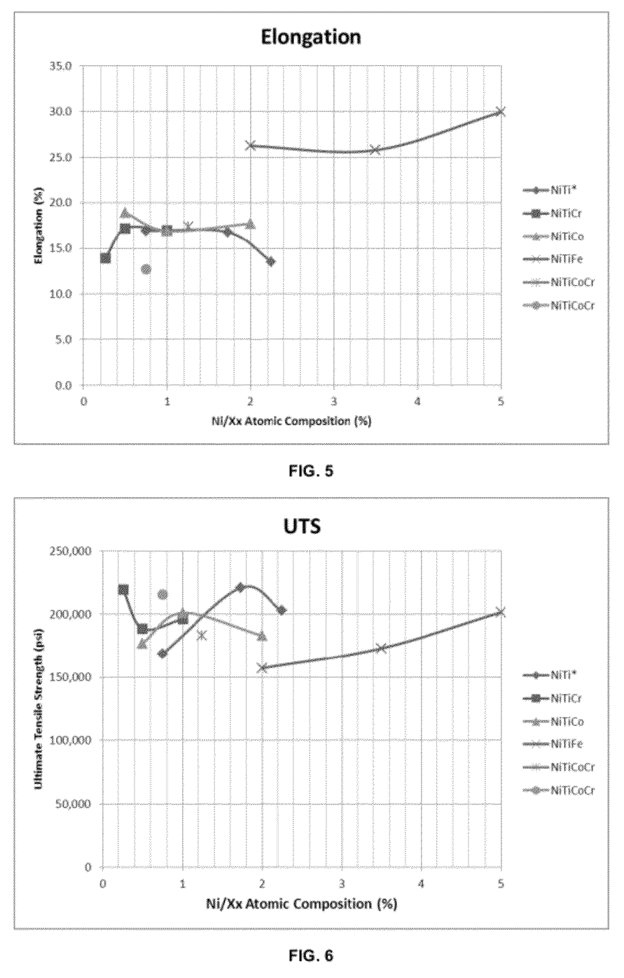

FIG. 5 is a plot of uniform elongation as a function of alloy composition for the tested binary Ni—Ti, Ni—Ti—Cr, Ni—Ti—Co, Ni—Ti—Fe and Ni—Ti—Co—Cr alloy samples. Uniform elongation provides a measure of the ductility of a sample and may be defined as the % elongation of the specimen at the maximum force sustained just prior to necking or fracture, or both, during the tensile test.

The binary Ni—Ti alloy samples show a maximum elongation of about 17% over a nickel concentration range of about 49.8 at. % to about 50.7 at. %. Similarly, the Ni—Ti—Cr alloy samples show a maximum elongation of about 17% over a Cr concentration range of about 0.5 at. % to about 1 at. %. The Ni—Ti—Co alloy samples exhibit a maximum elongation at a Co concentration of 0.5 at. %, and the elongation decreases (from about 19% to about 17%) as the Co concentration approaches 1 at. %. The elongation of the quaternary Ni—Ti—Co—Cr samples is in the range of about 13% to about 17%.

FIG. 6 is a plot of ultimate tensile strength (UTS, or tensile strength) as a function of alloy composition for the tested binary Ni—Ti, Ni—Ti—Cr, Ni—Ti—Co, Ni—Ti—Fe and Ni—Ti—Co—Cr alloy samples. Ultimate tensile strength may be defined as the maximum load applied in breaking the specimen during the tensile test divided by the original cross-sectional area of the specimen.

For the binary Ni—Ti alloy samples, UTS reaches a maximum of about 221 ksi at a nickel concentration of about 50.7 at. %. The Ni—Ti—Cr alloy samples show a maximum UTS of about 219 ksi at a Cr concentration of about 0.25 at. %, and the Ni—Ti—Co alloy samples show a maximum UTS of about 201 ksi at a Co concentration of about 1 at. %. The quaternary Ni—Ti—Co—Cr alloy samples have UTS values in the range of from about 183 ksi to about 215 ksi.

TABLE 3
Compilation of Tensile Test Results

| | | Ultimate | Upper | Lower | | | |
|---|---|---|---|---|---|---|---|
| | | Tensile | Plateau | Plateau | Hysteresis | | |
| | | Strength | Strength | Strength | (UPS- | Elongation | Perm |
| Sample | Specimen | (psi) | (psi) | (psi) | LPS) (psi) | (%) | Set |
| Ni—Ti | 1 | 169,314 | 20,800 | −58 | 20,858 | 17.5 | 4.97 |
| Low Ni | 2 | 168,848 | 19,753 | −96 | 19,849 | 16.8 | 4.95 |
| (49.75 | 3 | 168,275 | 21,432 | −84 | 21,516 | 17.1 | 4.86 |
| at. % Ni) | 4 | 168,486 | 21,399 | −98 | 21,497 | 17 | 4.87 |
| | 5 | 168,328 | 21,949 | −76 | 22,025 | 16.3 | 4.89 |
| | Average | 168,650 | 21,066 | −82 | 21,149 | 16.9 | 4.91 |
| Ni—Ti | 1 | 226,108 | 103,866 | 40001 | 63,865 | 17.6 | 0.11 |
| Medium | 2 | 225,608 | 101,590 | 40,074 | 61,516 | 17.9 | 0.09 |
| Ni | 3 | 225,941 | 102,187 | 45,384 | 56,803 | 18 | 0.1 |
| (50.73 | 4 | 207,601 | 95,049 | 44,756 | 50,293 | 15.1 | 0.13 |
| at. % Ni) | 5 | 218,391 | 100,287 | 44,114 | 56,173 | 15.3 | 0.16 |
| | Average | 220,730 | 100,596 | 42,866 | 57,730 | 16.8 | 0.12 |
| Ni—Ti | 1 | 188,938 | 79,311 | 22,361 | 56,950 | 12 | 0.16 |
| High Ni | 2 | 206,995 | 76,734 | 22,460 | 54,274 | 13.9 | 0.28 |
| (51.25 | 3 | 200,399 | 77,863 | 23538 | 54,325 | 13.8 | 0.12 |
| at. % Ni) | 4 | 211,221 | 77,613 | 25,020 | 52,593 | 14 | 0.31 |
| | 5 | 205,756 | 78,737 | 24,922 | 53,815 | 13.9 | 0.12 |
| | Average | 202,662 | 78,052 | 23,660 | 54,391 | 13.5 | 0.2 |
| NiTi—0.25Cr | 1 | 231,143 | 89,918 | 46,593 | 43,325 | 14.4 | 0.06 |
| | 2 | 230,915 | 91,062 | 45,905 | 45,157 | 14.03 | 0.06 |
| | 3 | 232,027 | 91,806 | 47,788 | 44,018 | 14.27 | 0.11 |
| | Average | 231,362 | 90,929 | 46,762 | 44,167 | 14.23 | 0.08 |
| NiTi—0.5Cr | 1 | 189,221 | 94,938 | 41,024 | 53,914 | 16.5 | 0.16 |
| | 2 | 188,675 | 94,120 | 40,400 | 53,720 | 17.4 | 0.17 |
| | 3 | 188,051 | 94,401 | 40,718 | 53,683 | 17.1 | 0.16 |
| | 4 | 187,981 | 97,271 | 41,884 | 55,387 | 17.7 | 0.18 |
| | 5 | 188,322 | 92,483 | 43,672 | 48,811 | 17.3 | 0.16 |
| | Average | 188,450 | 94,643 | 41,540 | 53,103 | 17.2 | 0.17 |
| NiTi—1Cr | 1 | 196,397 | 86,326 | 33,948 | 52,378 | 16.6 | 0.1 |
| | 2 | 195,773 | 86,752 | 32,860 | 53,892 | 17.4 | 0.12 |
| | 3 | 195,770 | 87,987 | 33,203 | 54,784 | 17.3 | 0.1 |
| | 4 | 196,413 | 88,418 | 33,402 | 55,016 | 17.6 | 0.12 |
| | 5 | 196,609 | 88,255 | 33,793 | 54,462 | 15.9 | 0.11 |
| | Average | 196,193 | 87,548 | 33,441 | 54,106 | 17 | 0.11 |
| NiTi—0.5Co | 1 | 177,895 | 119,923 | 53,653 | 66,270 | 19.8 | 0.73 |
| | 2 | 176,513 | 114,164 | 57,830 | 56,334 | 18.5 | 0.63 |
| | 3 | 176,565 | 120,929 | 52,401 | 68,528 | 19.2 | 0.76 |
| | 4 | 176,581 | 115,886 | 58,005 | 57,881 | 18.2 | 0.61 |
| | 5 | 177,569 | 119,040 | 54,237 | 64,803 | 18.8 | 0.7 |
| | Average | 177,025 | 117,988 | 55,225 | 62,763 | 18.9 | 0.68 |
| NiTi—1Co | 1 | 201,541 | 92,129 | 43,709 | 48,420 | 16.7 | 0.12 |
| | 2 | 201,204 | 92,014 | 43,303 | 48,711 | 17.4 | 0.13 |
| | 3 | 201,023 | 92,325 | 43,760 | 48,565 | 17.4 | 0.13 |
| | 4 | 200,614 | 93,365 | 43,758 | 49,607 | 16.5 | 0.11 |
| | 5 | 200,461 | 92,852 | 43,430 | 49,422 | 16.6 | 0.12 |
| | Average | 200,969 | 92,537 | 43,592 | 48,945 | 16.9 | 0.12 |
| NiTi—2Co | 1 | 183,341 | 114,083 | 64,764 | 49,319 | 18 | 0.54 |
| | 2 | 182,748 | 109,226 | 66,243 | 42,983 | 18.6 | 0.61 |
| | 3 | 182,646 | 114,849 | 65,157 | 49,692 | 17.7 | 0.54 |
| | 4 | 183,130 | 108,861 | 64,996 | 43,865 | 17.1 | 0.52 |
| | 5 | 182,627 | 111,061 | 65,897 | 45,164 | 17.2 | 0.55 |
| | Average | 182,898 | 111,616 | 65,412 | 46,205 | 17.7 | 0.55 |
| NiTi—2Fe | 1 | 157,158 | 140,556 | 6705 | 133,851 | 26.1 | 2.73 |
| | 2 | 157,354 | 140,546 | 6239 | 134,307 | 25.5 | 2.72 |
| | 3 | 156,928 | 141,022 | 6332 | 134,690 | 25.3 | 2.78 |
| | 4 | 157,455 | 143,721 | 7525 | 136,196 | 27.5 | 2.77 |
| | 5 | 157,525 | 143,765 | 6211 | 137,554 | 26.8 | 2.76 |
| | Average | 157,284 | 141,922 | 6602 | 135,320 | 26.2 | 2.75 |
| NiTi—4Fe | 1 | 173,488 | 168,512 | −150 | 168,662 | 27.7 | 3.42 |
| | 2 | 172,877 | 167,492 | −152 | 167,644 | 26.1 | 3.37 |
| | 3 | 172,665 | 167,578 | −176 | 167,754 | 22.7 | 3.39 |
| | 4 | 173,038 | 167,443 | −151 | 167,594 | 26.2 | 3.3 |
| | 5 | 172,785 | 167,375 | −202 | 167,577 | 26.2 | 3.41 |
| | Average | 172,971 | 167,680 | −166 | 167,846 | 25.8 | 3.38 |
| NiTi—5Fe | 1 | 202,171 | 184,248 | −38 | 184,286 | 31.7 | 3.58 |
| | 2 | 201,695 | 183,760 | −59 | 183,819 | 28.4 | 3.67 |
| | 3 | 200,399 | 181,872 | −175 | 182,047 | 30.3 | 3.66 |
| | 4 | 202,038 | 184,296 | −185 | 184,481 | 29.3 | 3.65 |
| | 5 | 201,053 | 182,815 | −167 | 182,982 | 29.9 | 3.64 |
| | Average | 201,471 | 183,398 | −125 | 183,523 | 29.9 | 3.64 |
| NiTi—0.5Co—0.25Cr | 1 | 214,620 | 118,598 | 73,066 | 45,532 | 12.3 | N/R |
| | 2 | 215,209 | 118,743 | 73,967 | 44,776 | 12.5 | N/R |
| | 3 | 216,365 | 116,940 | 73,585 | 43,355 | 12.9 | 0.05 |
| | 4 | 215,224 | 119,321 | 71,488 | 48,833 | 12.5 | N/R |
| | 5 | 214,876 | 120,515 | 72,046 | 48,469 | 12.6 | 0.01 |
| | 6 | 215,213 | 119,591 | 71,737 | 47,854 | 13.6 | 0.08 |
| | 7 | 215,335 | 120,307 | 72,885 | 47,422 | 12.9 | N/R |
| | Average | 215,263 | 119,145 | 72,682 | 46,606 | 12.7 | 0.05 |
| NiTi—0.75Co—0.5Cr | 1 | 183,000 | 134,000 | 77,000 | 67,000 | 17.4 | 0.71 |

TABLE 4
Results of wire fatigue testing conducted on strain annealed wire

| Alloy | Endurance strain (%) |
|---|---|
| NiTi | 0.4 |
| NiTi—0.25Cr | 0.6 |
| NiTi—0.5Co—0.25Cr | 0.55 |
| NiTi—0.75Co—0.5Cr | 0.82 |

The tensile test results for the ternary Ni—Ti—X alloys set forth in the preceding table were analyzed as set forth above, along with data from differential scanning calorimetry (DSC) experiments, in order to propose an optimal quaternary nickel-titanium alloy composition, as described below. These data were compiled prior to melting the quaternary alloys of the compositions indicated in Table 1. Table 4 includes the results of wire fatigue testing carried out on the strain annealed wire samples. The quaternary Ni—Ti—Co—Cr specimens exhibit a significantly improved endurance strain values compared to the binary Ni—Ti sample. Advantageously, the endurance strain of the Ni—Ti—Co—Cr specimens is at least about 0.6%, and preferably at least about 0.7% or at least about 0.8%.

The quaternary nickel-titanium alloy includes Ni at a concentration of between about 48 at. % and about 52 at. %; Cr at a concentration of between about 0.3 at. % and about 1 at. %; Co at a concentration of between about 0.5 at. % and about 2 at. %; and Ti at a concentration wherein a ratio of Ni:Ti is about 1.025. The Cr concentration may also be between about 0.4 at. % and 0.6 at. %, and the Co concentration may be between about 0.6 at. % and about 0.9 at. %. According to a preferred embodiment, the concentration of Ni is between about 50.4 at. % and about 50.8 at. %; the concentration of Cr is about 0.5 at. %; and the concentration of Co is about 0.75 at. %.

The quaternary alloy has a desirable combination of mechanical and superelastic properties. In particular, the alloy has an upper plateau strength of at least about 100 ksi (preferably at least about 115 ksi or at least about 130 ksi) and a hysteresis of at least about 50 ksi (preferably at least about 55 ksi, or at least about 60 ksi), where the hysteresis is the difference between the upper plateau strength and a lower plateau strength. The alloy further exhibits a permanent set of less than about 0.2% (preferably less than about 0.1%) and a percent elongation of at least about 17%. The ultimate tensile strength is at least about 180 ksi (preferably at least about 200 ksi).

The phase transformation temperatures of the quaternary alloy include an austenite finish temperature, A_{f}, at or below body temperature (37° C.). Preferably, the A_{f} may be in the range of from about −15° C. to about 37° C. Even more preferably, the A_{f} may be in the range of from about −15° C. to about 20° C. An austenite start temperature (Aₛ) of the nickel-titanium alloy is preferably in the range of from about −25° C. to about 20° C., according to one embodiment. Advantageously, the quaternary alloy has a suppressed martensite start temperature compared to a binary nickel-titanium alloy. The martensite start temperature may be about −50° C. or below, and a martensite start temperature of about −80° C. or below is preferred. The quaternary nickel-titanium alloy may also include an intermediate temperature R-phase in addition to the higher temperature austenitic phase and the lower temperature martensitic phase.

The designer quaternary alloy set forth herein may have properties advantageous for medical devices. Accordingly, a medical device may include at least one component comprising the quaternary nickel-titanium alloy, which may include Ni at a concentration of from about 48 at. % to about 52 at. %; Cr at a concentration of from about 0.3 at. % to about 1 at. %; Co at a concentration of from about 0.5 at. % to about 2 at. %; and Ti at a concentration wherein a ratio of Ni:Ti is about 1.025. The concentration of Ni may be from about 50.4 at. % to about 50.8 at. % (e.g., about 50.7 at. %). In addition, the concentration of Cr may be from about 0.4 at. % to about 0.6 at. % (e.g., about 0.5 at. %); and the concentration of Co may be from about 0.6 at. % to about 0.9 at. % (e.g., about 0.75 at. %). According to another embodiment, the concentration of Cr may be about 0.3 at. % and the concentration of Co may be about 0.5 at. %The component may be formed in whole or in part of the quaternary nickel-titanium alloy from wire, tubing, ribbon, button, bar, disk, sheet, foil, or another cast or worked shape, such as a cannula. According to one embodiment, the component has a composite structure in which one or more portions of the structure are formed of the quaternary alloy, and one or more portions of the structure are formed of a different material. For example, the component may include distinct constituents, such as layers, cladding, filaments, strands, cables, particles, fibers, and/or phases, where one or more of the constituents are formed from the quaternary alloy, and one or more are formed from the different material.

The component comprising the quaternary alloy may be employed individually or in combination as part of an insertable or implantable medical device, such as, for example, a stent, a stent graft, a wire guide, a radiopaque marker or marker band, a torqueable catheter, an introducer sheath, an orthodontic arch wire, or a manipulation, retrieval, or occlusive device such as a grasper, a snare, a basket (e.g., stone extraction or manipulation basket), a vascular plug, or an embolic protection filter.

The nickel-titanium alloy is preferably biocompatible to ensure successful use in the human body as part of a medical device. When introduced into a patient, a biocompatible material or device will not cause an adverse reaction or response in a majority of the patients. The biocompatibility of the nickel-titanium alloy may be assessed according to American Society for Testing and Materials (ASTM) standards F748-04 entitled “Standard Practice for Selecting Generic Biological Test Methods for Materials and Devices,” F813-01 entitled “Standard Practice for Direct Contact Cell Culture Evaluation of Materials for Medical Devices,” and/or F895-84 entitled “Standard Test Method for Agar Diffusion Cell Culture Screening for Cytotoxicity.” Additionally, the International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing” may be useful in evaluating the biocompatibility of the nickel-titanium alloy and/or a medical device comprising the alloy. The aforementioned standards set forth practices and methods designed for evaluating cytotoxicity, infectivity, pyrogenicity, irritation potential, reactivity, hemolytic activity, carcinogenicity and/or immunogenicity, and are hereby incorporated by reference. Since biocompatibility is a function of the type of bodily tissue contact and the duration of contact, the amount of testing required generally depends on the application. For example, the biocompatibility testing requirements for a short term contacting basket are substantially different from those of a permanently implanted stent.

Cytotoxicity tests using the ISO elution method were carried out on one of the quaternary Ni—Ti alloys (50.6 at. % Ni, 0.5 at. % Co, 0.25 at. % Cr, balance Ti), which had been electropolished and passivated. The guidelines of “ISO 10993-5, Biological evaluation of medical devices—Part 5: Tests for in vitro cytotoxicity,” were followed. A single preparation of the test article was extracted in single strength Minimum Essential Medium (IX MEM) at 37° C. for 24 hours. The negative control, reagent control, and positive control were similarly prepared. Triplicate monolayers of L-929 mouse fibroblast cells were dosed with each extract and incubated at 37° C. in the presence of 5% CO, for 48 hours. Following incubation, the monolayers were examined microscopically for abnormal cell morphology and cellular degeneration. The test article showed no evidence of causing cell lysis or toxicity. The test article extract met the requirements of the test since the grad was less than a grade 2 (mild reactivity).

To produce the nickel-titanium alloys of the present disclosure and medical devices comprising the alloys, a melt including the desired amounts of alloying elements is formed and then cooled into a solid (e.g., an ingot). High purity raw materials (e.g., Ti>99.7 wt. % purity and Ni>99.99 wt. % purity) are preferably melted in an inert gas or vacuum atmosphere.

Melting methods known in the art, including but not limited to vacuum induction melting (VIM), vacuum consumable arc melting (VAR), and electron beam melting, may be employed to form the melt. Remelting is generally desirable to obtain satisfactory microstructural homogeneity in the ingot. For example, successive VAR processes or a VIM/VAR double melting process may be employed.

The ingot may then be hot worked into a first shape (e.g., bar, rod, tube hollow, or plate) by, for example, extruding, hot rolling, or forging. Hot working is generally employed to refine the cast structure of the ingot and to improve mechanical properties. The hot working is generally carried out at temperatures in the range of from about 700° C. to about 950° C., and may require multiple hot working and reheating cycles. The reheating may be carried out over an eight hour period, for example. Preferably, the ingot undergoes a minimum deformation of about 90% during hot working in order to homogenize the as-cast, dendritic microstructure. Prior to hot working, it may be beneficial to carry out a solution heat treatment that involves soaking the ingot at an elevated temperature for a given time duration, followed by quenching. The solution heat treatment may aid in homogenizing the microstructure of the alloy and may be carried out at a temperature in the range of from about 850° C. to about 1150° C., for example.

The first shape (e.g., bar, rod, tube, or plate) may then be cold worked into a component by cold drawing or cold rolling, for example. The cold working typically involves several passes in combination with interpass annealing treatments at temperatures in the range of from about 600° C. to about 800° C. The interpass annealing treatments soften the material through recrystallization and growth of the austenite grains between cold work passes, where 30-40% deformation is typically imparted. If cold drawing is employed to form a wire, for example, a polycrystalline diamond die with a molybdenum disulphide or other suitable lubricant may be employed in order to reduce the drawing stress. Machining operations, such as, for example, drilling, cylindrical centerless grinding, or laser cutting may also be employed to fabricate the component. Other operations, such as wire braiding or winding, may also be carried out.

A heat treatment is employed to impart a “memory” of a desired final shape and to optimize the shape memory/superelastic and mechanical properties of the component. The number, duration and the temperature of the heat treatments may alter the transformation temperatures. Typically, heat treatment temperatures of 350° C. to 550° C. are appropriate to set the final shape and optimize the shape memory/superelastic and mechanical properties. Preferably, the heat treating involves annealing the component while constrained in a final shape at a temperature in the range of from about 350° C. to about 550° C. More preferably, heat treatment or annealing temperatures in the range of from 450° C. to 550° C. are appropriate.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A quaternary nickel-titanium alloy comprising:
Ni at a concentration of from about 48 at. % to about 52 at. %;
Cr at a concentration of from about 0.3 at. % to about 1 at. %;
Co at a concentration of from about 0.5 at. % to about 2 at. %; and
Ti at a concentration wherein a ratio of Ni:Ti is about 1.03.

2. The alloy of claim 1 comprising a suppressed martensite start temperature compared to a binary nickel-titanium alloy.

3. The alloy of claim 2, wherein the martensite start temperature is about −80° C. or below.

4. The alloy of claim 1 comprising an upper plateau strength of at least about 100 ksi.

5. The alloy of claim 4, wherein the upper plateau strength is at least about 115 ksi.

6. The alloy of claim 4 comprising a hysteresis of at least about 50 ksi, the hysteresis being a difference between the upper plateau strength and a lower plateau strength.

7. The alloy of claim 6, wherein the hysteresis is at least about 55 ksi.

8. The alloy of claim 1 comprising a permanent set of less than about 0.2%.

9. The alloy of claim 1 comprising a percent elongation of at least about 17%.

10. The alloy of claim 1 comprising an ultimate tensile strength of at least about 200 ksi.

11. The alloy of claim 1 having an austenite finish temperature at or below about 37° C.

12. The alloy of claim 1 wherein:
the concentration of Cr is from about 0.4 at. % to about 0.6 at. %; and
the concentration of Co is from about 0.6 at. % to about 0.9 at. %;

13. The alloy of claim 12, wherein:
the concentration of Cr is about 0.5 at. %; and
the concentration of Co is about 0.75 at. %.

14. The alloy of claim 1 wherein:
the concentration of Cr is about 0.25 at. %; and
the concentration of Co is about 0.5 at. %.

15. The alloy of claim 1, wherein:
the ratio of Ni:Ti is about 1.025 and the concentration of Ni is from about 50.4 at. % to about 50.8 at. %.

16. A medical device comprising at least one component comprising a quaternary nickel-titanium alloy including Ni at a concentration of between about 48 at. % and about 52 at. %; Cr at a concentration of between about 0.3 at. % and about 1 at. %; Co at a concentration of between about 0.5 at. % and about 2 at. %; and Ti at a concentration wherein a ratio of Ni:Ti is about 1.03.

17. The medical device of claim 16, wherein the component includes at least one of a wire and a cannula.

18. The medical device of claim 16, wherein:
the concentration of Cr is from about 0.4 at. % to about 0.6 at. %; and
the concentration of Co is from about 0.6 at. % and 0.9 at. %;

19. The medical device of claim 18, wherein:
the concentration of Cr is about 0.5 at. %; and
the concentration of Co is about 0.75 at. %.

20. The medical device of claim 16, wherein:
the concentration of Cr is about 0.25 at. %; and
the concentration of Co is about 0.5 at. %.
